(19) United States
(12) Patent Application Publication
(10) Pub. No.: US 2017/0224774 A1
(43) Pub. Date: Aug. 10, 2017

(54) METHOD FOR USING SUPPLEMENTAL VASCULAR ENDOTHELIAL GROWTH FACTOR (VEGF) OR ANALOG TO PREVENT OXYGEN INDUCED ARREST OF VESSEL GROWTH AND DISEASE SEQUELA OF PREMATURE INFANT BIRTH

(71) Applicant: Kenneth W. Wright, Los Angeles, CA (US)

(72) Inventor: Kenneth W. Wright, Los Angeles, CA (US)

(21) Appl. No.: 15/425,872

(22) Filed: Feb. 6, 2017

Related U.S. Application Data
(60) Provisional application No. 62/292,537, filed on Feb. 8, 2016.

(51) Int. Cl. A61K 38/18; A61B 3/14; A61B 5/02; A61K 9/00

(52) U.S. Cl. CPC A61K 38/1866; A61K 9/0019; A61B 3/14; A61B 5/02028

(57) ABSTRACT

Disclosed herein is a method for administering systemic (e.g., by intravenous injection) supplemental vascular endothelial growth factor (VEGF) to premature infants to prevent the disease sequelae and complications including but not limited to retinopathy of prematurity (ROP) and/or paraventricular leukomalacia (PVL) and/or bronchopulmonary dysplasia (BPD) associated with premature births. The VEGF is administered to the premature infant to produce a physiologic serum VEGF level that is substantially similar to the VEGF level found in a normally developing fetus in the utero. By way of one example, the retinal blood vessel growth of the premature infant is monitored, and the administration of VEGF is terminated when the infant's retinal blood vessels are substantially fully grown to ROP Zone 3. By virtue of the method herein disclosed, normal vessel growth occurs, and lung disease, ROP with blindness, brain damage and other diseases associated with premature birth are substantially avoided.

### CROSS REFERENCE TO RELATED PATENT APPLICATIONS

This application is related to Provisional Patent Application No. 62/292,537 filed Feb. 8, 2016.

### BACKGROUND OF THE INVENTION

1. Field of the Invention

This invention relates to a method for using supplemental vascular endothelial growth Factor (VEGF) soon after birth to prevent the disease sequelae and complications of premature birth Oxygen given to the premature it reduces systemic and tissue VEGF levels which arrests normal vessel growth, causes vascular involution (vaso-obliteration), and causes the disease sequelae and complications of premature birth. By maintaining physiologic VEGF levels with the early administration of systemic VEGF, normal vessel growth occurs thus preventing the complications of premature birth including but not limited to lung disease, eye disease and brain damage.

2. Background Art

Major disease sequelae and complications of premature birth include but not limited to retinopathy of prematurity (ROP), bronchopulmonary dysplasia (BPD), and periventricular leukomalacia (PVL). These sequelae are most severe and prevalent in very premature infants under 1,250 grams birth weight, ROP can cause blindness from retinal scarring. BPD is known to cause lung disease that reduces lung capacity making infants dependent on supplemental oxygen. PVL is known to cause brain damage and delayed motor development, cerebral palsy, seizures, cortical visual impairment and visual perceptive problems.

A premature infant is really a fetus out of the womb. In utero, the fetus is in a very low oxygen environment (physiologic hypoxia) with arterial partial pressure of oxygen around 2 mm hg. This physiologic hypoxia is vital as low blood and tissue oxygen tension induce the production of the important hormone VEGF (Vascular Endothelial Growth Factor), VEGF stimulates normal vessel growth and is necessary for maintenance of vessel integrity. VEGF is produced when tissue oxygen levels are low and VEGF production is down regulated when tissue oxygen levels are high. In the developing fetus the low oxygen environment causes VEGF levels to be high (serum about 550 pg/ml) in blood and tissue as compared to adult VEGF levels (serum about 150 pg/ml). High fetal VEGF levels are critical for stimulating normal blood vessel growth in the rapidly growing fetus. VEGF has many forms including VEGF-A, VEGF-B, VEGF-C, VEGF-D, VEGF-E and placenta growth factor (PIGF). VEGF-A is the strongest stimulator of vessel growth and promotes vessel survival. VEGF-A is usually called VEGF, with many isoforms including 121, 145, 165, 189, 206. Therefore, VEGF as used hereinafter refers to VEGF-A and other forms thereof.

Premature birth exposes the fetus now out of the womb to a ambient air and supplemental oxygen causing abnormally high blood and tissue oxygen levels. High systemic oxygen levels down regulates VEGF production and blood and tissue VEGF levels become abnormally low. Low tissue and blood VEGF levels arrest blood vessel growth and cause shrinkage of existing vessels commonly referred to as vaso-obliteration. This reduces the blood supply to developing tissues which is known to cause damage in many tissues including but not limited to the lungs, retina and brain.

In the retina, lack of blood supply from vaso-obliteration over several months causes chronic tissue hypoxia (ischemia) and results in a late secondary increase in retinal production of VEGF. This late increase in retinal VEGF stimulates the damaged retinal vessels to sprout abnormal new vessels called neovascularization or proliferative ROP. The abnormal new vessels leak protein and bleed causing retinal hemorrhage and scarring which results in retinal detachment and can cause blindness. The standard treatment of proliferative ROP is laser ablation of the hypoxic retina to reduce VEGF overproduction or intraocular in of anti-VEGF drugs such as bevacizumab (Avastin®). Thus, the treatment of proliferative ROP is anti-VEGF treatment. Even with anti-VEGF treatment, retinal, scarring from proliferative ROP can occur causing irreversible blindness.

Clinical studies by the applicant herein and others have shown that restricting supplemental oxygen at birth and avoiding hyperoxia reduces the incidence of proliferative ROP and PVL in premature infants. However, lower supplemental oxygen therapy has resulted in increased mortality because of a lack of oxygen. Therefore, many neonatologists now are concerned about using low oxygen protocols. A major difficulty in every neonatal intensive care unit (NICU) is regulating, supplemental oxygen for premature infants as too much oxygen will down regulate VEGF resulting in arrest of vessel growth and vaso-obliteration causing ROP, BPD, and PVL. On the other hand, not enough oxygen can cause hypoxic brain damage and increased mortality. It is therefore appreciated that there is a long felt need for a method and or drug to prevent the devastating sequelae and complications of premature birth including but not limited to ROP, PVL, and, BPD and allow the safe administration of oxygen to premature infants.

### SUMMARY OF THE INVENTION

In general terms, a method is disclosed herein for using supplemental or a VEGF analog to prevent the disease sequels and complications of premature infant birth including but not limited to ROP and/or PVL and/or BPD. Supplemental VEGF or VEGF analog is administered systemically (e.g., intravenously) to the premature infant to produce physiologic systemic VEGF levels in the premature infants substantially equal to the VEGF levels found in the fetus in the uterus. The VEGF is provided immediately after birth and fetal physiologic levels of VEGF are maintained until vessel growth is completed. Maintaining the VEGF levels similar to physiologic fetal levels stimulates vessel growth important in the developing and growing premature infant. Maintaining adequate systemic VEGF levels will protect the premature infant from the ill effects of oxygen and prevent to complications of premature birth. Early treatment with systemic VEGF soon after birth and before the occurrence of oxygen induced vaso-obliteration prevents the disease sequela and complications of premature birth including but not limited to lung tissue, and or retinal tissue and or brain tissue. This early VEGF treatment and maintenance of adequate blood and tissue VEGF levels promotes normal vessel growth and prevents vaso-obliteration. In the retina, supplemental VEGF prevents retinal ischemia thus preventing subsequent late secondary up regulation of retinal VEGF and proliferative ROP.

Monitoring vessel growth and vessel maturation can be accomplished by ophthalmoscopy and by serial retinal exams to document peripheral vessel growth. When retinal vessel growth is complete or near complete (to ROP Zone 3) then VEGF can be tapered and or stopped. Adequate VEGF levels means the maintenance of VEGF levels in the premature infant similar to VEGF levels found in the normal fetus. Adequate systemic VEGF levels, stimulates premature infant vessel growth even in the presence of relative hyperoxia from ambient air or supplemental oxygen. By virtue of providing adequate systemic VEGF levels in the premature infant, supplemental oxygen can be administered to the premature infant with less chance of oxygen induced vaso-obliteration. Maintenance of fetal physiologic levels of VEGF advantageously prevents oxygen induced vaso-obliteration thus encouraging overall infant growth and preventing the disease sequelae of premature birth including but not limited to ROP, BPD, and PVL.

### BRIEF DESCRIPTION OF THE DRAWINGS

There are no drawings with this application.

### DESCRIPTION OF THE PREFERRED EMBODIMENT

Disclosed herein is a method for preventing the disease sequelae and complications of premature birth including, but not limited to ROP, and/or PVL, and/or BPD and for allowing the safe administration of supplemental oxygen to premature infants. The method includes the step of administering systemic supplemental vascular endothelial growth factor (VEGF) or isoforms thereof or a VEGF analog or recombinant VEGF to a premature infant at or soon alter birth to maintain physiologic levels of VEGF that are substantially similar to VEGF levels found in the normal fetus in the womb. Administering supplemental systemic VEGF to premature infants counters the oxygen induced down regulation of VEGF caused by exposure to the relative high oxygen environment from ambient air or supplemental oxygen given to premature infants. Providing and maintaining physiologic fetal serum VEGF levels eliminates a reduction in the infant's blood vessel growth, avoids vaso-obliteration commonly caused by hyperoxia induced down regulation of VEGF, and prevents the pathological sequelae of premature birth including ROP, and/or BPD, and/or PVL. Providing supplemental VEGF allows an administration of oxygen without encountering the ill effect of reducing blood and tissue VEGF thus reducing the risk of hypoxic brain damage and death.

Supplemental VEGF is administered systemically such as by means of an intravenous, intermuscular, subcutaneous, transdermal, or intraperitoneal injection or other systemic route. The VEGF dose administered will be based on birth weight, infant maturity, and blood VEGF levels. VEGF is given in an amount to maintain physiologic serum VEGF levels similar to that found in the normal developing fetus around 550 pg/ml. Serum VEGF levels will be monitored, and fetal physiologic blood levels of VEGF are maintained by multiple doses or continuous IV infusion. Supplemental VEGF is stopped when the premature infant's blood vessel growth matures. The timing of the administration of preventative treatment with supplemental VEGF is important as free VEGF has a short half-life around 90 minutes. VEGF is given within hours after birth and before fetal serum VEGF levels fall from oxygen induced down regulation of VEGF, thus preventing the arrest of vessel growth and vaso-obliteration. Therefore, prior to the administration of the VEGF, it is desirable to keep the delivery of supplemental oxygen low so as to stimulate vessel growth. Because of the short half-life of VEGF supplemental VEGF could be given several times a day, by continuous IV infusion, or other means such as a transdermal patch,

The method herein disclosed solves the need for being able to prevent the devastating sequelae and complications of premature birth and allowing the safe administration of supplemental oxygen to premature infants. In this regard, it may be appreciated that the preventive treatment step of administering supplemental VEGF to a premature infant is in fact counter intuitive to the treatment of proliferative ROP associated with premature birth. In that case, the common treatment is anti-VEGF drug injections into the eye which, as will be known to those skilled in the art, is opposite to the step of administering VEGF in the manner described above. In fact, it is not known to the applicant to use of VEGF in clinical situations for treating disease in premature infants.

As an additional optional step to the method described above for using supplemental VEGF to prevent disease sequels and complications in premature infants, other growth factors can be simultaneously administered in addition to VEGF. Such additional growth factors to encourage vessel growth in the infant include insulin growth factor, IGF-1, insulin growth factor binding protein (IGFBP), or an analog thereof. Such additional growth factors will ideally be administered systemically (e.g., intravenously) during/with the administration of the supplemental VEGF.

An additional step to the method described above is using ophthalmoscopy to visualize the retina and monitor vessel growth and the state of vessel maturity. Other methods for visualizing the retina include ophthalmic photography and retinal imaging. Observing peripheral vessel growth provides information regarding outcome of VEGF treatment. State of retinal vessel maturation and retinal vascularization is a determinative factor as to when retinal vessel growth is complete or near complete (ROP Zone 3) and when supplemental VEGF can be tapered and or stopped. When the retina is fully vascularized or nearly vascularized supplemental (ROP Zone 3) this is termed mature retinal vascularization and then VEGF can be tapered and or stopped.

1. A method for preventing the disease sequels and complications of premature birth including but not limited to retinopathy of prematurity (ROP) and/or paraventricular leukomalacia (PVL) and/or bronchopulmonary dysplasia (BPD), comprising the step of administering to a premature infant after birth vascular endothelial growth factor (VEGF) for producing a physiologic serum VEGF level in the premature infant which is substantially similar to the systemic VEGF level of the infant lying in utero.

2. The method for preventing disease sequels and complications as recited in claim 1, comprising the additional step of administering the VEGF to the premature infant intravenously.

3. The method for preventing disease sequels and complications as, recited in claim 1, comprising the additional steps of administering the VEGF to the premature infant closely following its birth and prior to the fetal serum VEGF level of the premature infant falling from the oxygen induced down regulation of VEGF, and continuing the administration of the VEGF as is necessary to maintain the premature infant's fetal physiologic serum level of VEGF substantially similar to that found in a normally developing fetus lying in utero.

4. The method for preventing disease sequels and complications as recited in claim 1, comprising the additional step of administering the VEGF to the premature infant prior to the occurrence of oxygen induced vaso-obliteration.

5. The method for preventing disease sequels and complications as recited in claim 1, comprising the additional steps of delivering relatively low supplemental oxygen to the premature infant after birth and increasing the delivery of the supplemental oxygen to the premature infant only after the step of administering the VEGF.

6. The method for preventing disease sequels and complications as recited in claim 1, comprising the additional step of administering to the premature infant after birth a growth factor for the same duration of time during which the VEGF is administered.

7. The method for preventing disease sequels and complications as recited in claim 1, comprising the additional steps of monitoring the retinal blood vessel growth of the premature infant and terminating or tapering the administration of the VEGF when the premature infant's retinal blood vessels are mature and substantially fully grown to ROP Zone 3.

8. The method for preventing disease sequels and complications as recited in claim 7, comprising the additional step of monitoring the retinal blood vessel growth of the premature infant by means of visualizing the retina of the premature infant.

9. The method for preventing disease sequels and complications as recited in claim 8, comprising the additional step of using at least one of ophthalmoscopy, ophthalmic photography, and retinal imaging to visualize the retina of the premature infant.
